(19) United States
(12) Patent Application Publication
(10) Pub. No.: US 2026/0306008 A1
(43) Pub. Date: Oct. 8, 2026

(54) EARPIECE FOR A WEARABLE ELECTRONIC DEVICE AND METHOD OF MANUFACTURING THE SAME

(71) Applicant: T&W Engineering A/S, Alleroed (DK)

(72) Inventors: Niels Christian Damgaard JAKOBSEN, Vaerloese (DK); Klaus Henrik VAARBROE, Skovlunde (DK); John LOUGART, Roskilde (DK)

(73) Assignee: T&W Engineering A/S, Alleroed (DK)

(21) Appl. No.: 19/169,334

(22) Filed: Apr. 3, 2025

(51) Int. Cl. A61B 5/369; A61B 5/00; H04R 31/00

(52) U.S. Cl. CPC A61B 5/369; A61B 5/6817; A61B 2562/12; H04R 31/00

(57) ABSTRACT

An earpiece to be worn in the ear, for a wearable electronic device, in particular an EEG logger or a hearing instrument. The earpiece has an earpiece body of an elastic material and designed to be inserted into the ear thereby abutting an inner surface of the ear. The earpiece also has at least one electronic sensor or actor fixed to the earpiece body, and at least one isolated electric wire electrically connected, at one end thereof, to the at least one sensor or actor. At least one lug is integrally formed to the earpiece body, the at least one lug having a through-hole through which the wire passes. In a relaxed state, the through-hole has a width being smaller than an outer width of the wire such that the wire expands the lug, when passing through the through-hole causing the lug to exert an elastic force to the wire.

### FIELD AND BACKGROUND OF THE INVENTION

The invention relates to an earpiece for a wearable electronic device, in particular an EEG logger or a hearing instrument, which earpiece is configured to be worn in the ear of a (human) user of the wearable electronic device. The invention also relates to a method of manufacturing said earpiece.

In general, the present invention relates to small electronic devices designed to be operated when being worn at the body of a (human) person, which person is called the user or wearer of the wearable electronic device.

One specific type of a wearable electronic device to which the present invention relates, is an EEG logger. An EEG logger, as disclosed and claimed herein, is a wearable electronic device for monitoring electroencephalogram (EEG) data, i.e. bio-electric brain waves of a human user (patient). Typically, such devices are adapted for long term monitoring, e.g. several days, months or even years, where the person can live a normal life without having to pay more attention to the monitoring than necessary with a pair of glasses or a hearing aid. The monitoring may be for purposes of surveillance of a condition of the person and/or for providing an alarm or information in case predetermined conditions are met. The monitor may also be applied for collection of data for further analysis, e.g. for diagnostic purposes or for research use.

As an example of monitoring EEG signals for various diagnostic purposes, WO 2006/047874 A1 describes measurement of brain waves particularly for detecting the onset of an epileptic seizure.

EEG loggers may also be applied for surveillance of persons having diabetes, where low blood sugar levels may cause hypoglycemic attacks. A system for surveillance of the EEG signal where changes may indicate an imminent hypoglycemic attack is disclosed in WO 2006/066577 A1. Especially for long term monitoring, it is known to make use of implants to pick up the bio-electrical signals. These implants feature a transmission unit to send the picked-up signals to a recording unit. Such a system is known for example from WO 2015/144214 A1. Other EEG loggers have electrodes that can be removably attached to the body of the user.

Another specific type of a wearable electronic device to which the present invention relates, is a hearing instrument. In general, a hearing instrument is an electronic device being designed to support the hearing of a person wearing it. In particular, the invention relates to hearing aids, i.e., hearing instruments that are specifically configured to at least partially compensate a hearing impairment of a hearing-impaired user. Hearing instruments are most often designed to be worn in or at the ear of the user, e.g., as a Behind-The-Ear (BTE) or In-The-Ear (ITE) device. With respect to its internal structure, a hearing instrument normally comprises an (acousto-electrical) input transducer, a signal processor, and an output transducer. During operation of the hearing instrument, the input transducer captures a sound signal from an environment of the hearing instrument and converts it into an input audio signal (i.e., an electrical signal transporting a sound information). In the signal processor, the input audio signal is processed, in particular amplified dependent on frequency, e.g., to compensate the hearing-impairment of the user. The signal processor outputs the processed signal (also called “output audio signal”) to the output transducer. Most often, the output transducer is an electro-acoustic transducer (also called “receiver”) that converts the output audio signal into a processed air-borne sound which is emitted into the ear canal of the user. Alternatively, the output transducer may be an electro-mechanical transducer that converts the output audio signal into a structure-borne sound (vibrations) that is transmitted, e.g., to the cranial bone of the user. Besides classical hearing aids, there are hearing instruments (sometimes referred to as “Personal Sound Amplification Products”) that are designed to support the hearing of normal-hearing users (i.e., users without hearing impairment), in particular to protect the hearing in harmful sound environments or to allow for better speech perception in complex background noise.

Both types of wearable electronic devices (as well as other wearable electronic devices, e.g. smart phones, portable music players, in-ear monitors for musicians, etc.) may comprise or be used in combination with at least one earpiece to be worn in one of the ears of the user, which earpiece typically includes at least one electronic sensor or actor. In the case of an EEG logger, the at least one earpiece typically includes one or more of the EEG logger's electrodes that detect the bio-electric brain waves of the user. In the case of a hearing instrument (as in the case of music players or smart phones), the earpiece typically includes the (electro-acoustic or electro-mechanical) output transducer. In addition, the earpiece of a hearing instrument may include one or more sensors, e.g. at least one microphone, a pulse monitor, etc.

Such earpieces often include an earpiece body made of an elastic (i.e. flexibly deformable) material that is designed to abut against an inner surface of the ear, e.g. a wall of the concha and/or the ear canal, when the earpiece is inserted in the ear. In particular, the earpiece body may be an ear mold that is individually adapted to the inner shape of the user's ear and designed to snuggly fit to the wall of the concha and/or the ear canal. Moreover, such earpieces often include at least one (electrically) isolated electric wire for transferring at least one electric signal between the at least one sensor or actor of the earpiece and an electronic main unit of the wearable electronic device.

### SUMMARY OF THE INVENTION

An object of the present invention is to provide an earpiece as described above that is reliable and easy to manufacture. A related object of the present invention is to provide an easy-to-realize method of manufacturing a reliable earpiece as described above.

In embodiments of the present invention, an earpiece for a wearable electronic device, for example an EEG logger or a hearing instrument, is provided, which earpiece is designed to be worn in one of the ears of a user. The earpiece comprises an earpiece body made of an elastic (i.e. flexibly deformable) material. It is designed to be inserted into the ear (i.e. the concha and/or the ear canal) such that it abuts against an inner surface of the ear (i.e. against an inner wall of the concha and/or the ear canal). In preferred embodiments of the invention, the earpiece body is an ear mold that is individually adapted to snuggly fit into the user's ear. The earpiece further comprises at least one electronic sensor or actor being fixed to the earpiece body, i.e. fixedly arranged, in particular embedded in the earpiece body. The earpiece also comprises at least one (electrically) isolated electric wire which is electrically connected, at one end, to the at least one sensor or actor. For example, said at least one isolated electric wire may be configured to transfer at least one electric signal between the at least one sensor or actor and an electronic main unit of the wearable electronic device.

In order to fixedly attach the at least one isolated wire to the earpiece body, at least one lug is integrally (i.e. monolithically) formed from the earpiece body. Said at least one lug has a through-hole through which the wire passes (i.e. protrudes). Herein, the term “through-hole” denotes a hole that extends over the entire width of the lug and opens to two opposite sides of the lug.

In a relaxed state of the lug (i.e. without the wire being inserted into the through-hole), said through-hole has a width being smaller than a corresponding outer width of the wire such that the wire expands the lug, when it is inserted in the through-hole. Herein, expansion of the elastic material of the lug causes the lug to exert an elastic (radial) force to the wire to lock the latter against an axial movement relative to the lug. Herein, the term “width” generally denotes the distance between opposite portions of a wall of the through-hole or wire. For through-holes or wires having a circular cross-section, the “width” is identical to the diameter of said cross-section.

It has been found that the configuration described above provides a particularly easy-to-realize way of safely fixing the at least one wire to the earpiece body, assuring a stable fixation of the at least one wire in a well-defined position. Thereby, damage or early wearout of the wire in use of the earpiece can be avoided effectively.

The fixation of the at least one wire by lugs formed integrally from the flexible earpiece body has been found to be particularly beneficial for flexible earpiece bodies as described above, and superior to other fixation methods; herein, a particular problem is that conventional flexible materials being suitable for manufacture of the earpiece body typically have a low friction coefficient which makes a mechanic connection of the at least one isolated wire with the earpiece body difficult to realize. For example, the present inventors found that embedding the at least one wire in the material of the earpiece body during manufacture is difficult to realize with satisfactory results as a well-defined and reproducible position of the wire in the earpiece body can hardly be assured with reasonable effort. The flexible material of the earpiece body showed a strong tendency to push the isolated wire to the surface of the earpiece body, during manufacture of the latter, where the wire was exposed and susceptible to damage during use of the earpiece. The present inventors also tried to guide the at least one wire in a groove inserted in the material of the earpiece body and to fix the at least one wire in said groove by gluing. However, safely fixing the wire by gluing was found to be difficult, due to the low friction coefficient of the earpiece body. Moreover, the glue negatively affected the desired flexibility of the earpiece body. Finally, loosely routing the at least one wire along the earpiece body was also tried but found to render the earpiece susceptible to damage.

In preferred embodiments of the invention, the earpiece body is formed (in particular monolithically) from an elastomer such as silicone. Preferably, the at least one isolated electric wire has an (electrically) isolating coating that may be made of a polymer material. In different embodiments of the invention, e.g., said isolating coating may be made of Polytetrafluoroethylene (PTFE), Polyvinyl chloride (PVC), Polyurethane (PU), Polyphenyl ether (PPE), in particular Modified polyphenyl ether (MPPE), or a Perfluoroalkoxy alkane (PFA).

In easy-to-realize but effective embodiments of the invention, the through-hole in the at least one lug is made after manufacturing a raw form of the earpiece body, using a material-removing processing such as drilling, stamping or cutting. In particular, laser-drilling or laser cutting may be used to produce the through-hole in the at least one lug.

Preferably, in its relaxed state, said through-hole has a width (in particular, diameter) between 90% and 98%, preferably between 92% and 96%, and in particular 94% of the corresponding outer width (in particular, outer diameter) of the isolated wire. In a specific example, in its relaxed state, said through-hole has a diameter of 0.50 mm, whereas the isolated wire has an outer diameter of 0.53 mm.

In preferred embodiments of the invention, the earpiece is attached to or integrated into in a connecting part, which is configured to mechanically and/or electrically connect the earpiece to another earpiece to be worn at the other ear of the user and/or to the electronic main unit of the wearable electronic device. In particular, said connecting part may be provided to guide the at least one isolated electric wire or an extension thereof from the earpiece to the electronic main unit of the wearable electronic device. The connecting part may be realized, e. g, as a flexible cable or an elastic head or neck band, and may be fixedly attached to the earpiece and/or the electronic main unit or detachable from both the at least one earpiece and the electronic main unit.

In general, the manufacturing method according to the invention is provided and designed for manufacture of the earpiece according to the invention (as described above). Any embodiment and variation of the earpiece described above corresponds to a embodiment and variation of the method. Thus, disclosure provided above as to features of the earpiece and related benefits can be applied to corresponding features of the method, mutatis mutandis, and vice-versa.

In the method for manufacturing the earpiece, a raw form of said earpiece body is manufactured using a forming process, in particular resin injection molding, casting or diecasting. Herein, the raw form is made with at least one protrusion designed to form (i.e. to be later processed into) said at least one lug. Thereafter, said through-hole is made into the at least one protrusion, using material-removing processing, to transform the at least one protrusion into the said least one lug. For example, one of drilling, stamping and cutting (in particular laser drilling or laser-cutting) is used to produce the through-hole.

In an alternative embodiment of the method, the earpiece body is made by 3D-printing. Herein, preferably, the earpiece body is 3D-printed in its final state, i.e. with the respective through-hole being established ab initio in the lug or each of the lugs. Alternatively, within the scope of the invention, the earpiece body may be 3D-printed as a raw form (as described above) and the or each through-hole may be produced thereafter, using material-removing processing as described above.

For inserting the at least one isolated wire in the through-hole, a (hollow) guide tube is pushed through the through-hole thereby expanding the material of said at least one lug and, thus, increasing the width of the through-hole. Said at least one electrical wire is then inserted in the guide tube such that the wire passes (extends) through the through-hole. Finally, the guide tube is removed from the through-hole causing the lug to contract and exert the elastic (radial) force to the wire. The lug thereby locks the wire by friction against an axial movement in relation to the lug.

In preferred embodiments of the invention, a plurality of lugs (as described above) are formed from the earpiece body. In particular, a series of several lugs may be provided to guide a same isolated wire along a pre-defined path on the earpiece body. In these cases, the methods steps described above are executed repeatedly for any through-hole of said plurality of lugs. In further embodiments, the earpiece may include two or more isolated electrical wires being connected to the same sensor or actor and/or, if applicable, to different sensors and/or actors of the earpiece. In these cases, any of said two or more isolated wires may be fixed to the earpiece body separately by one respective lug or the sequence of lugs, as described above. As an alternative, a plurality of wires may be fixed side-by-side by the same lug or series of lugs.

In further steps, preferably, the at least one isolated wire is attached to or inserted in the connecting part and electrically connected to the at least one sensor or actor. Both steps can be executed after passing the at least one isolated wire through the lug (or any of the plurality of lugs, if applicable). In alternative implementations of the method, one of said further steps can be executed before inserting the at least one isolated wire in said at least one lug.

Moreover, before or after inserting the at least one isolated wire in said at least one lug, the sensor or actor is attached to the earpiece body, in particular inserted in a corresponding receptacle of the earpiece body.

As mentioned above, in preferred embodiments of the invention, the wearable electronic device to which the earpiece is attributed, may be a EEG logger. Preferably, said EEG logger comprises a head section to be worn at the head of a user. The EEG logger further comprises a main unit. The head section includes a plurality of electrodes (i.e. at least two electrodes, but preferably at least three electrodes, for example five or six electrodes) for measuring EEG signals, i.e. electric voltage signals corresponding to bio-electric brain waves. Herein, the head section comprises or is attached to at least one earpiece as described above wherein said earpiece carries at least one of said electrodes.

Preferably, the EEG logger includes a housing, a controller and a rechargeable battery. The controller is arranged in the housing of the main unit and adapted to control said electrodes and to record said EEG signals. The battery is also arranged in the housing of the main unit and provided for supplying electric power to the controller. By preference, the controller is formed by or comprises at least one programmable unit such as a microcontroller. In this case, at least some of the functions of the EEG logger are implemented as software installed in a form being executable by the controller. Alternatively, the controller may be realized as a non-programmable hardware circuitry, e.g. as an ASIC. Moreover, the controller may comprise at least one programmable and at least one non-programmable sub-unit.

Different from some of the known EEG loggers mentioned above that include an implanted measuring unit, preferably, the present invention relates to a wearable EEG logger, the head section of which is not implanted but worn at the head similar to a pair of glasses or a headphone. It can thus be removed from the head easily by hand.

In preferred embodiments, the head section comprises two earpieces that can be inserted into the ears (in particular the concha) of the user, and a band connecting said two earpieces, similar to the band of an earphone. Optionally, at least one of the electrodes be arranged in the band connecting the earpieces. In accordance with the invention, said band may be designed to be worn on top of the head. However, in preferred embodiments, the band is designed as a neck band that is worn in the neck or behind the head of the user.

### BRIEF DESCRIPTION OF THE FIGURES

FIG. 1 shows a perspective view of an EEG logger comprising a head section and a detachable main unit with the main unit being attached to a connector housing of the head section;

FIG. 2 shows a perspective view as in FIG. 1 of the EEG logger with the main unit being detached from the connector housing of the head section;

FIG. 3 shows a perspective view of an earpiece of the EEG logger attached to an ear hook section of a neck band of the head section; and

FIG. 4-7 shows a perspective view similar to FIG. 3 of four consecutive states during manufacture of the earpiece.

### DETAILED DESCRIPTION OF THE INVENTION

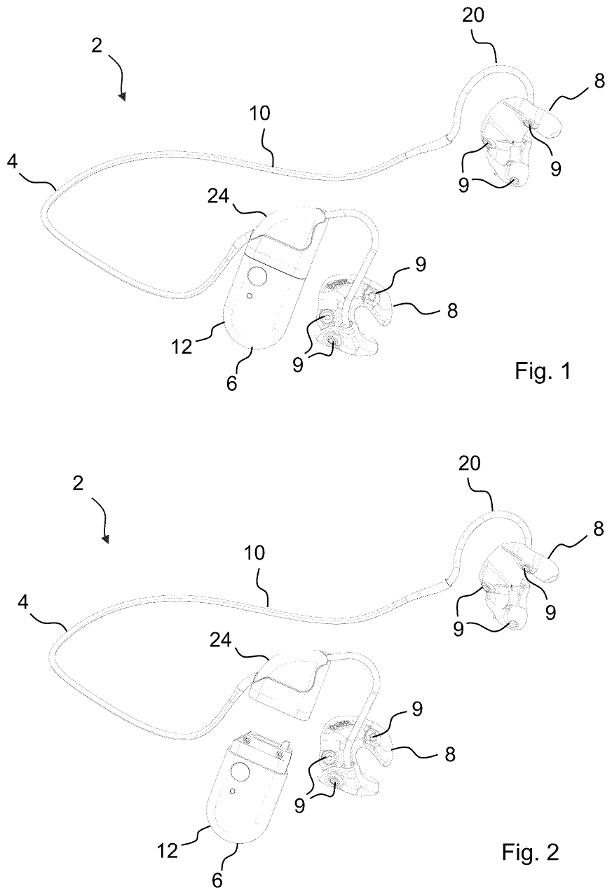

Referring now to the figures of the drawings in detail and first, particularly to FIG. 1 thereof, there is shown an embodiment of a wearable an EEG logger 2, i.e. a wearable electronic device for monitoring electroencephalogram (EEG) data, i.e. bio-electric brain waves of a human user (patient). The EEG logger 2 comprises a head section 4 to be worn at the head of a user and a main unit 6. The head section 4 includes two earpieces 8 that can be inserted into the ears (each in a respective concha) or the user. The head section 4 also includes a connecting part that mechanically connects said two earpieces 8 with each other and with the main unit 6. In the shown embodiment, said connecting part is designed as a neck band 10 that is worn in the neck or behind the head of the user.

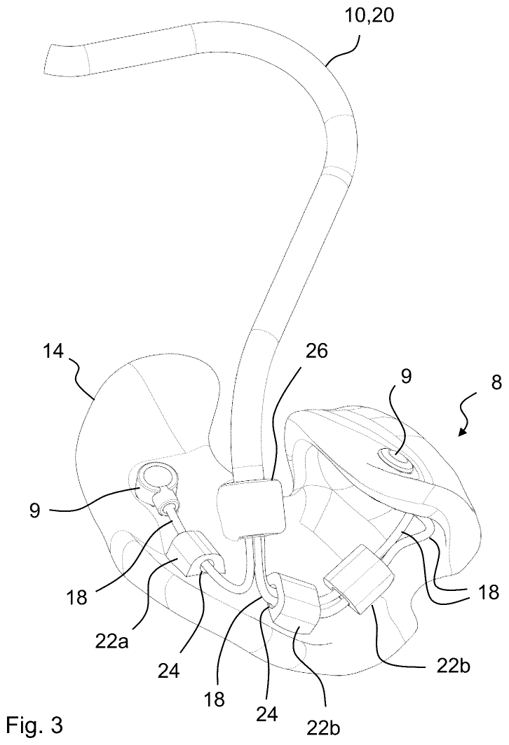

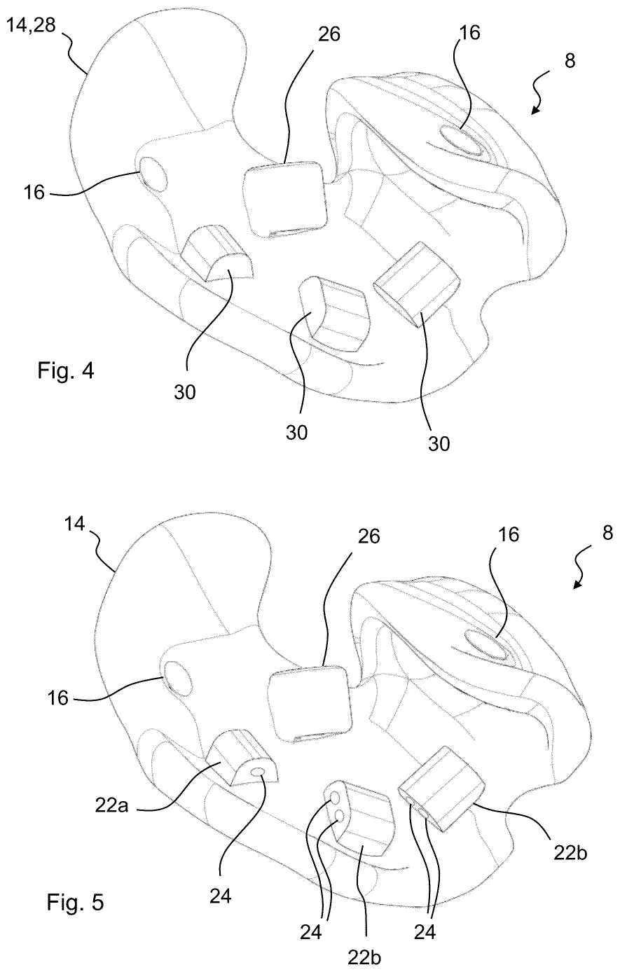

As is best seen in FIG. 3, the head section 4 includes a plurality of electrodes 9 (e.g. six electrodes 9) for measuring EEG signals, i.e. electric voltage signals corresponding to bio-electric brain waves. In the example shown in FIGS. 1 and 2, said electrodes 9 are arranged at the earpieces 8 (e.g. 3 electrodes 9 at each one of the earpieces 8). Optionally, further ones of said plurality of electrodes 9 may be arranged at the neck band 10.

The main unit 6 includes a housing 12 in which, i.a, a controller and a rechargeable battery are located. Preferably, the controller is formed by a (programmable) microcontroller with a memory in which an executable software (firmware) is installed. The controller may also include an ASIC configured to read-out the EEG signals from the electrodes 9 and to convert these signals into digital data. By virtue of the firmware, the controller is adapted to record the EEG signals measured by the electrodes 9. The battery is provided for supplying electric power to the controller.

In operation, the controller receives the EEG signals from the electrodes 9 via separate wired connections between each of the electrodes 9 and the controller. The controller is configured to record (i.e. to at least temporarily store) the received EEG signals for later evaluation by a health care professional, to transfer the received EEG signals wirelessly to a remote evaluation unit and/or to evaluate the received EEG data in real time, e.g. in order to detect imminent or otherwise unrecognized epileptic seizures.

The main unit 6 is detachably connected to the head section 4 by a releasable connection. In particular, the main unit 6 is detached from the head section 4 for charging the battery and/or for reading out the recorded EEG signal (cf. FIG. 2).

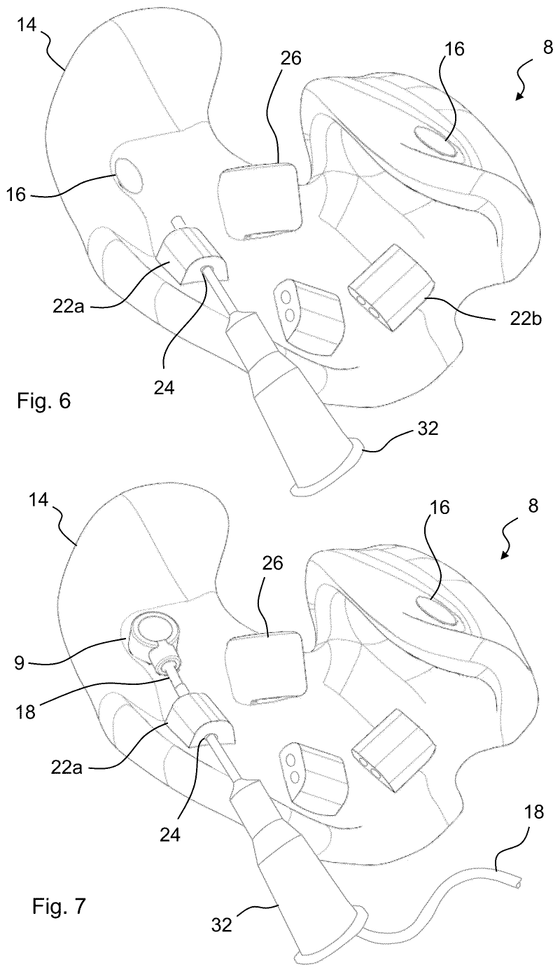

As can be seen, e.g., from FIG. 3, each one of the earpieces 8 is designed as an ear mold being individually adapted to the shape of the ear of the user. Each of the earpieces 8 comprises an earpiece body 14 made of an elastic material and designed to be inserted into the ear thereby abutting against an inner surface of the ear (more precisely the concha) of the user. The earpiece body 14 has three apertures 16 (or through-holes, see, e.g., FIG. 4) which are provided as receptacles into each of which one of the electrodes 9 is inserted. Each of the electrodes 9 is electrically connected to an (electrically) isolated electric wire 18 (or, more precisely, a pair of wires in a common protective coating), by which the respective electrode 9 is electrically connected to the main unit 6. In a preferred implementation, the protective coating of the wires 18 is made of Polytetrafluoroethylene (PTFE). The wires 18 of each earpiece 8 are guided to a ear hook section 20 of the neckband 10 that guides the wires 18 to the main unit 6.

In order to fixedly attach the wires 18 at the earpiece body 14 and to ensure a well-defined path of each wire 18 along an inner surface of the respective earpiece 8, the earpiece body 14 is provided with a number of (in the shown example three) lugs 22a, 22b, wherein each of said lugs 22a, 22b has at least one through-hole 24 through which one of the wires 18 protrudes. More precisely, the earpiece body 14 is provided with one lug 22a having one through-hole 24 through which one of the wires 18 protrudes, and two further lugs 22b in a serial arrangement, wherein each of said further lugs 22b has two through-holes 24 (or one through-hole 24 of larger size) through which two further wires 18 are passed. The three wires 18 are, thus, guided to a sleeve 26 of the earpiece 8, in which an end of a corresponding one of said ear hook sections 20 of the neck band 10 is inserted (and, thus, fixed to the earpiece body 14). At their respective distal ends (as seen from the main unit 6), each of the wires 8 is electrically connected to one of the electrodes 9 which are inserted in the apertures 16 (and, thus, fixed to the earpiece body 14). The lugs 22a, 22b are integrally (i.e. monolithically) formed from the earpiece body 14 and are, thus, made of elastic material of the latter. Likewise, the sleeve 26 is integrally (i.e. monolithically) formed from the earpiece body 14. In a suited implementation, the earpiece body 14 is made of silicone.

Contrary to what the simplified representation of FIG. 3 seems to show, each of the through-holes 24 has a smaller diameter as compared to the outer diameter of the corresponding wire 18, when the corresponding lug 22a, 22b is in a relaxed state (i.e. before the wire 18 or other part is inserted into the through-hole 24). In embodiments in which the lugs 22b are provided with one through-hole designed to receive two wires 18, said through-hole is designed with dimensions smaller than the width of the combined pair of wires 18. Thus, when being inserted into the through-hole 24, the wire 18 expands (or the wires 18 expand) the respective lug 22a, 22b. In response, due to the elasticity of the lugs'22a, 22b material, the lugs 22a, 22b exert an elastic (radial) force to the wires 18 that locks the wires 18 against axial movement relative to the lugs 22a, 22b.

The FIGS. 4 to 7 show different successive states of the earpiece body during manufacture of the earpiece 8.

In a first step, a raw form 28 of the earpiece body 14 (shown in FIG. 4) is manufactured using a forming process, in particular by resin injection molding, casting or diecasting. The raw form 28 is made with three protrusions 30 at an inner side of the raw form 28, wherein each of said protrusions 30 is a raw form of a later lug 22a or 22b.

In a subsequent step, the through-holes 24 are made in the protrusions 30 using material-removing processing such as drilling, stamping and cutting (in particular laser drilling or laser-cutting), to thereby transform the protrusions 30 into the lugs 22a, 22b. FIG. 5 shows the earpiece body 14 with the lugs 22a, 22b being provided with the through-holes 24.

In alternative embodiments of the method, the earpiece body 14 is made by 3D-printing, either by 3D-printing the earpiece body 14 in its final state or by 3D-printing the raw form 28 of the earpiece body 14 with subsequent drilling of the through-holes 24.

In a further step, in order to insert one of the wires 18 into a corresponding through-hole 24 of one of the lugs 22a, 22b a guide tube 32 is pushed through the through-hole 24 (FIG. 6). The guide tube 32 has an inner diameter that exceeds the outer diameter of the wire 18 to be inserted. Thus, necessarily, the outer diameter of the guide tube 32 exceeds the width of the through-hole 24 and expands the lug 22a, 22b, when it is pushed into the through-hole 24.

Now, the wire 18 is inserted into the guide tube 32, as shown in FIG. 7. Thereafter, the guide tube 32 is carefully pulled out of the through-hole 24 while retaining the wire 18, causing the lug 22a, 22b to contract and exert said elastic force to the wire 18. Finally the guide tube 32 is removed from the wire 18.

These steps are repeated for every through-hole 24 starting with the through-holes 24 that are located closest to the apertures 16. Thus, the wires 18 are laid between the apertures 16 and the sleeve 26, via one or more lugs 22a, 22b.

In further steps, before, between or after the steps mentioned above, as appropriate, the electrodes 8 are inserted into their apertures 16, the end of the ear hook section 20 is inserted into the sleeve 26 and the wires 18 are threaded through the ear hook section 20 and the neck band 10 to the main unit 6.

It will be appreciated by persons skilled in the art that numerous variations and/or modifications may be made to the invention as shown in the specific examples without departing from the spirit and scope of the invention as broadly described in the claims. The present examples are, therefore, to be considered in all aspects as illustrative and not restrictive.

The following is a summary list of reference numerals and the corresponding structure used in the above description of the invention:
2 EEG logger
4 head section
6 main unit
8 earpiece
9 electrode
10 neck band
12 housing
14 earpiece body
16 apertures
18 wire
20 ear hook section
22a, 22b lug
24 through-hole
26 sleeve
28 raw form
30 protrusion
32 guide tube

1. An earpiece to be worn in the ear of a user, for a wearable electronic device, in particular an EEG logger or a hearing instrument, the earpiece comprising:
an earpiece body made of an elastic material and designed to be inserted into the ear thereby abutting against an inner surface of the ear;
at least one electronic sensor or actor being fixed to the earpiece body;
at least one isolated electric wire electrically connected, at one end thereof, to the at least one sensor or actor;
wherein at least one lug is integrally formed to the earpiece body, said at least one lug having a through-hole through which the wire passes;
wherein, in a relaxed state thereof, said through-hole has a width being smaller than an outer width of the wire such that the wire expands the lug, when passing through the through-hole causing the lug to exert an elastic force to the wire.

2. The earpiece as claimed in claim 1, wherein the earpiece body is formed from an elastomer, in particular silicone.

3. The earpiece as claimed in claim 1, wherein the at least one isolated electric wire has an isolating coating made of a polymer material, in particular one of Polytetrafluoroethylene, Polyvinyl chloride, Polyurethane, Polyphenyl ether or a Perfluoroalkoxy alkane.

4. The earpiece as claimed in claim 1, wherein the through-hole in the at least one lug is made after manufacturing a raw form of the earpiece body, by material-removing processing, in particular drilling, stamping or cutting.

5. The earpiece as claimed in claim 1, wherein, in its relaxed state, said through-hole has a width between 90% and 98%, preferably between 92% and 96%, and in particular 94% of the corresponding outer width of the wire.

6. The earpiece as claimed in claim 1, being attached to or integrated in a connecting part which is configured to mechanically and/or electrically connect the earpiece to another earpiece to be worn at the other ear of the user and/or to the electronic main unit of the wearable electronic device.

7. A wearable electronic device, in particular an EEG logger or a hearing instrument, comprising:
an electronic main unit to be worn at the body of a user;
at least one earpiece to be worn in the ear of a user; and
a connecting part being configured to transfer at least one electric signal between the at least one sensor or actor of the earpiece and the electronic main unit;
wherein said at least one earpiece includes an earpiece body made of an elastic material and designed to be inserted into the ear thereby abutting against an inner surface of the ear; at least one electronic sensor or actor being fixed to the earpiece body; at least one isolated electric wire electrically connected, at one end thereof, to the at least one sensor or actor;
wherein at least one lug is integrally formed to the earpiece body, said at least one lug having a through-hole through which the wire passes; and
wherein, in a relaxed state thereof, said through-hole has a width being smaller than an outer width of the wire such that the wire expands the lug, when passing through the through-hole causing the lug to exert an elastic force to the wire.

8. The wearable electronic device as claimed in claim 7, wherein the earpiece body is formed from an elastomer, in particular silicone.

9. The wearable electronic device as claimed in claim 7, wherein the at least one isolated electric wire has an isolating coating made of a polymer material, in particular one of Polytetrafluoroethylene, Polyvinyl chloride, Polyurethane, Polyphenyl ether or a Perfluoroalkoxy alkane.

10. The wearable electronic device as claimed in claim 7, wherein the through-hole in the at least one lug is made after manufacturing a raw form of the earpiece body, by material-removing processing, in particular drilling, stamping or cutting.

11. The wearable electronic device as claimed in claim 7, wherein, in its relaxed state, said through-hole has a width between 90% and 98%, preferably between 92% and 96%, and in particular 94% of the corresponding outer width of the wire.

12. A method of manufacturing an earpiece to be worn in the ear of a user, for a wearable electronic device, in particular an EEG logger or a hearing instrument, the earpiece including an earpiece body made of an elastic material and designed to be inserted into the ear thereby abutting against an inner surface of the ear; at least one electronic sensor or actor being fixed to the earpiece body; and at least one isolated electric wire electrically connected, at one end thereof, to the at least one sensor or actor, said method comprising:
manufacturing a raw form of said earpiece body using a forming process, in particular resin injection molding, casting or diecasting, said raw form having at least one protrusion;
making a through-hole into the at least one protrusion, using material-removing processing, in particular drilling, stamping or cutting to transform the at least one protrusion into at least one lug;
pushing a guide tube through the through-hole thereby expanding the material of said at least one lug:
inserting said at least one electrical wire in the guide tube such that the wire passes through the through-hole; and
removing the guide tube from the through-hole causing the lug to exert an elastic force to the wire.

13. The method as claimed in claim 12, wherein the earpiece body is formed from an elastomer, in particular silicone.

14. The method as as claimed in claim 12, wherein the at least one isolated electric wire has an isolating coating made of a polymer material, in particular one of Polytetrafluoroethylene, Polyvinyl chloride, Polyurethane, Polyphenyl ether or a Perfluoroalkoxy alkane.

15. The method as claimed in claim 12, wherein, in its relaxed state, said through-hole has a width between 90% and 98%, preferably between 92% and 96%, and in particular 94% of the corresponding outer width of the wire.
